(19) United States
(12) Patent Application Publication
(10) Pub. No.: US 2012/0172907 A1
(43) Pub. Date: Jul. 5, 2012

(54) Tissue Removal Apparatus

(75) Inventors: Natasha Lee Shee, Maple (CA); Neil Godara, Milton (CA); Padina Pezeshki, Toronto (CA); Caitlyn Paget, Toronto (CA)

(73) Assignee: Kimberly-Clark, Inc., Mississauga (CA)

(21) Appl. No.: 12/981,651

(22) Filed: Dec. 30, 2010

(51) Int. Cl. A61B 17/00

(52) U.S. Cl. 606/190

(57) ABSTRACT

An apparatus is disclosed for removing tissue from a patient's body. The apparatus comprises a first elongate member defining a lumen and having an open distal end. The open distal end defines an opening and comprises a tip, where the tip is structured to reduce damage to tissue within the patient's body. The apparatus further comprises a tissue removal member disposed within the lumen of the elongate member. The tissue removal member comprises a distal end that is recessed from the tip of the elongate member. At least a portion of the tissue removal member protrudes beyond the opening to allow access to tissue distal to the opening. Related hollow members with blunt tips are also disclosed.

### TECHNICAL FIELD

The present disclosure relates to devices for removal of material from a patient's body. More particularly, the disclosure relates to such devices that are minimally invasive.

### BACKGROUND

Various devices have been proposed for removing tissue from a desired area within a patient. Such devices often include a tube such as a catheter, lumen, etc., guided to a site of interest and a device of some sort located within the tube to remove tissue from the site of interest.

For example, Hamatura et al. in U.S. Pat. No. 6,554,799, issued Apr. 29, 2003 discloses a biological precision screw pump capable of transferring a sufficient amount of viscous liquid even with a very thin suction and injection pipe. The disclosure provides a pump capable of minimizing invasion into human bodies by housing a very thin rotor in a cylindrical needle, and positively transferring a liquid based on the mechanical configuration of the rotor. A viscous liquid can be moved by increasing the number of rotations of the rotor and that the pipe diameter can be reduced by twisting up a plurality of thin filaments to obtain a rotor

Cooke et al. in U.S. Pat. No. 6,926,725 discloses an improvement to a thrombectomy apparatus for breaking up thrombus or other obstructive material in a lumen of a vascular graft or vessel. The wire is operatively connected to a motor for rotation of the wire to enable peaks of the sinuous wire to contact a wall of the lumen to break up the thrombus or other obstructive material. The apparatus comprises a wire being formed of an inner core formed by a plurality of twisted wires and an outer wire wound directly around the inner core. The tightly wound inner/outer core structure enable rotation of the distal of the wire corresponding to rotation at its proximal end as torque is transmitted to its distal end.

U.S. Pat. No. 5,041,082 to Shiber issued Aug. 20, 1991 discloses a mechanical atherectomy system insertable into a patient's artery over a non-rotating, auger shaped flexible guide-wire. A portion of the length of the flexible guide-wire located near the front end is shaped as an auger which is formed by a spaced spiral-wire attached to a core-wire. Once the flexible guide-wire is in place, the flexible rotary catheter and the tubular-blade are advanced to the obstruction site, and continue to be advanced into the obstruction while being rotated over the flexible guide-wire.

U.S. Pat. No. 6,758,851 to Shiber issued Jul. 6, 2004 discloses an apparatus for extracting an obstruction located in a patient's vessel. The apparatus has a flexible-tube with an open distal end that is connected to a negative pressure source. The apparatus further comprises flexible-tube containing a motor rotated conveyor-shaft to which an offset-agitator is connected. The direction of rotation of the conveyor-shaft's spiral is such that as it rotates relative to the flexible tube it conveys the fragments co-operatively with the negative pressure, from the open distal end through the flexible-tube. Shiber discloses that at least a part of the conveyor-shaft and preferably substantially all of its length is a spiral with gaps between its coils to enable the spiral to convey the fragments. The apparatus may be delivered to an obstruction site over a guide-wire

In U.S. Pat. No. 6,926,725, issued Dec. 13, 1988, Hawkins Jr. et al. disclose an apparatus and method for removing a target object from a body passageway. The apparatus comprises a catheter, a spiral wound coil disposed within the catheter and rotatably driven by an air actuated control means and a parachute basket. The spiral wound coil has a cutting tip at its distal end which is housed within the distal tip of the catheter. The target object is fragmented by the cutting action of the tip of the spiral wound coil as it is rotated at high speed within the catheter by the air actuated drive means. Rotation of the spiral wound coil also facilitates transport of the target fragments though the catheter lumen simultaneously with aspiration.

However, in each of the devices above, improvements cold be made in terms of providing a minimally invasive insertion wherein a tip of a tube such as a lumen is configured to reduce tissue damage during such insertion. Accordingly, an apparatus for removing tissue from a patient's body that achieved such reduction and/or addressed one or more other drawbacks of conventional devices would be welcome.

### SUMMARY

In one broad aspect, embodiments of the present disclosure comprise an apparatus for removing tissue from a patient's body. The apparatus comprises a first elongate member defining a lumen and having an open distal end, the open distal end defining an opening and comprising a tip, the tip being structured to reduce damage to tissue within the patient's body. The apparatus further comprises a tissue removal member disposed within the lumen, the tissue removal member having a distal end, the distal end being recessed from the tip, at least a portion of the tissue removal member protruding beyond the opening to allow access to tissue distal to the opening. Various options and modifications are possible.

For example, as a feature of this aspect, the open distal end comprises a bevel face, the bevel face being at an angle with respect to a longitudinal axis of the elongate member. In one example, the angle ranges from about 10 degrees to about 15 degrees. In another example, the angle is about 12.5 degrees.

As another feature of this aspect, the tip is a blunt tip. In one example the blunt tip is a square blunt tip. In another example, the blunt tip is a curved blunt tip. In another example, the blunt tip is a tapered blunt tip. In still another example, the blunt tip is a blunt lancet tip.

According to other aspects of the disclosure, a hollow member is provided for insertion into a patient's body to receive a tissue removal member for removing tissue via the hollow member. The hollow member comprises an elongate member defining a lumen and having an open distal end having a bevel face. The bevel face is at an angle with respect to a longitudinal axis of said elongate member. The open distal end defines an opening and has a blunt tip. The blunt tip is structured for advancement within the patient's body to the site of tissue to be removed. The presence of the blunt tip structure reduces damage to tissue within the patient's body during advancement. As above, various options and modifications are possible.

For example, the blunt tip may be square, curved, bevelled or a lancet. Also, the blunt tip may have a grit-blasted surface texture.

### BRIEF DESCRIPTION OF THE DRAWINGS

In order that the disclosure may be readily understood, embodiments of the disclosure are illustrated by way of examples in the accompanying drawings, in which:

FIG. 1A is a side perspective view of an elongate member;

FIG. 1B is a side perspective view of an elongate member with a bevel face;

FIG. 2A is a top-view of the distal end of an elongate member in accordance with an embodiment of the present disclosure;

FIGS. 2B is a side view of the distal end of an elongate member in accordance with an embodiment of the present disclosure;

FIG. 2C is a cross-sectional view of an elongate member taken along the line 2C-2C of FIG. 2B;

FIG. 2D is a side view of an elongate member in accordance with an embodiment of the present disclosure;

FIGS. 3A-3C show top perspective views of the distal end of an elongate member in accordance with different embodiments of the present disclosure;

FIG. 3D is a top view of the distal end of an elongate member in accordance with an alternate embodiment of the present disclosure;

FIG. 3E is a top perspective view of an elongate member in accordance with an embodiment of the present disclosure;

FIG. 4 is a side view of a tissue-removal apparatus in accordance with an embodiment of the present disclosure;

FIG. 5 is a top perspective view of the distal end of an elongate member with a tissue removal member disposed therein, in accordance with an embodiment of the present disclosure;

FIG. 6 is an illustration of a method in accordance with an embodiment of the present disclosure;

FIG. 7 is an illustration of a method in accordance with an embodiment of the present disclosure; and

FIG. 8 is an illustration of a method in accordance with an embodiment of the present disclosure.

### DETAILED DESCRIPTION

With specific reference now to the drawings in detail, it is stressed that the particulars shown are by way of example and for purposes of illustrative discussion of certain embodiments of the present disclosure only, and are presented in the cause of providing what is believed to be the most useful and readily understood description of the principles and conceptual aspects of the disclosure. In this regard, no attempt is made to show structural details of the disclosure in more detail than is necessary for a fundamental understanding of the disclosure, the description taken with the drawings making apparent to those skilled in the art how the several forms of the disclosure may be embodied in practice.

Before explaining at least one embodiment of the disclosure in detail, it is to be understood that the disclosure is not limited in its application to the details of construction and the arrangement of the components set forth in the following description or illustrated in the drawings. The disclosure is capable of other embodiments or of being practiced or carried out in various ways. Also, it is to be understood that the phraseology and terminology employed herein is for the purpose of description and should not be regarded as limiting.

As used herein, the term “coring” refers to advancing an elongate member defining a lumen and having an open distal end into a tissue, wherein the advancement results in the incorporation or gathering of at least a portion of the tissue into the lumen of the elongate member.

As used herein, the phrase “operatively connected” is intended to mean “coupled or connected, either directly or indirectly, such that the connected structures are operable to perform a desired function”.

As used herein, the term “conveyance” refers to facilitation of movement of a material from one location to another.

As used herein “blunt tip” refers to a tip that does not have a sharp edge or a point.

In one broad aspect, the present disclosure comprises an apparatus for removal of materials from the body of a patient. In one specific embodiment of the method, the apparatus is used for removal of nucleus pulposus tissue from an intervertebral disc. The apparatus may generally comprise a tissue removal member housed at least partially within an elongate member defining a lumen, for example a sheath. The tissue removal member may be any device that functions to convey tissue from the distal end of the elongate member to a portion exterior to the patient's body. In one embodiment, the tissue removal member is a shaft with projections extending outwardly from the shaft. In one embodiment, the tissue removal member is operatively connected to a motor or other source of rotational energy, which provides the motion required to remove the tissue. The elongate member has an open distal end with a bevel face. Furthermore, the elongate member has a blunt distal tip that is substantially atraumatic.

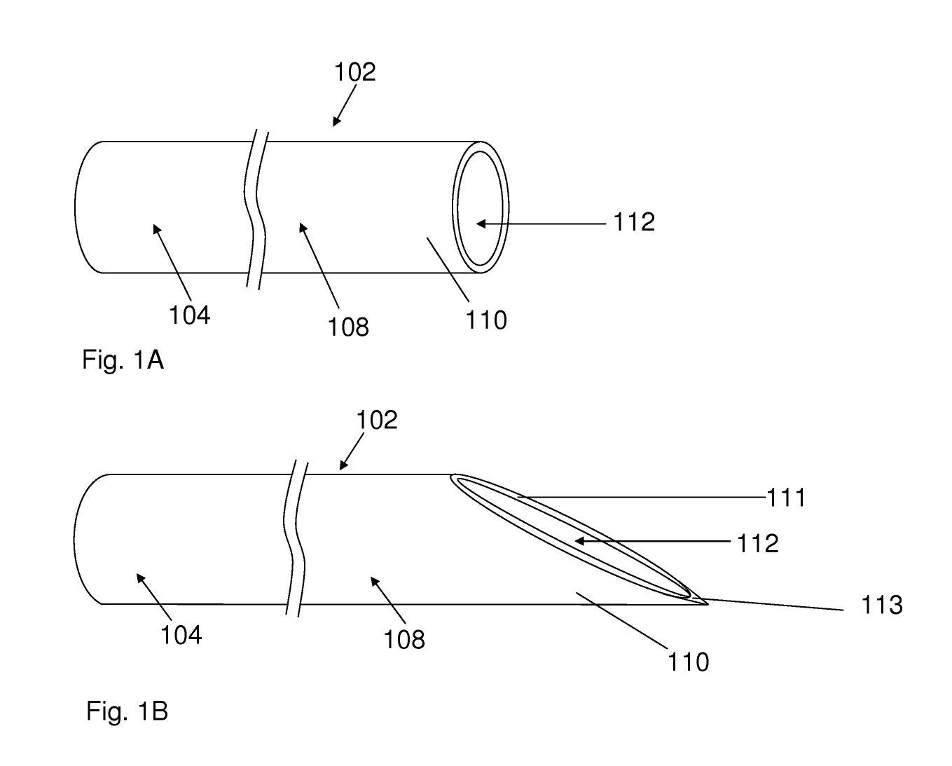

In the general embodiment shown in FIG. 1A, an elongate member 102 is disclosed that comprises a proximal portion 104 and distal portion 108 ending in an open distal end 110. The open distal end 110 defines an opening 112. In one specific embodiment, the cross-sectional shape of the elongate member 102 is substantially circular; however alternate embodiments are possible, wherein the shape may be ovoid, square, or rectangular and elongate member 102 is not limited in this regard. In the illustrated embodiment, the opening 112 is substantially perpendicular to the longitudinal axis of the shaft.

FIG. 1B illustrates a specific embodiment of the present disclosure where the open distal end 110 comprises a bevel face 111 defining an opening 112. The plane in which the opening or aperture 112 is defined may not be perpendicular to the longitudinal axis of elongate member. The bevel face 111 is cut at an angle with respect to the longitudinal axis of the elongate member 102. In one example the cut is performed using a laser. In an alternate example the elongate member 102 is cut using Electrical Discharge Machining (EDM). In alternate embodiments any other means of cutting may be used to form the bevel face 111. The creation of the bevel face 111 creates a sharp distal tip 113.

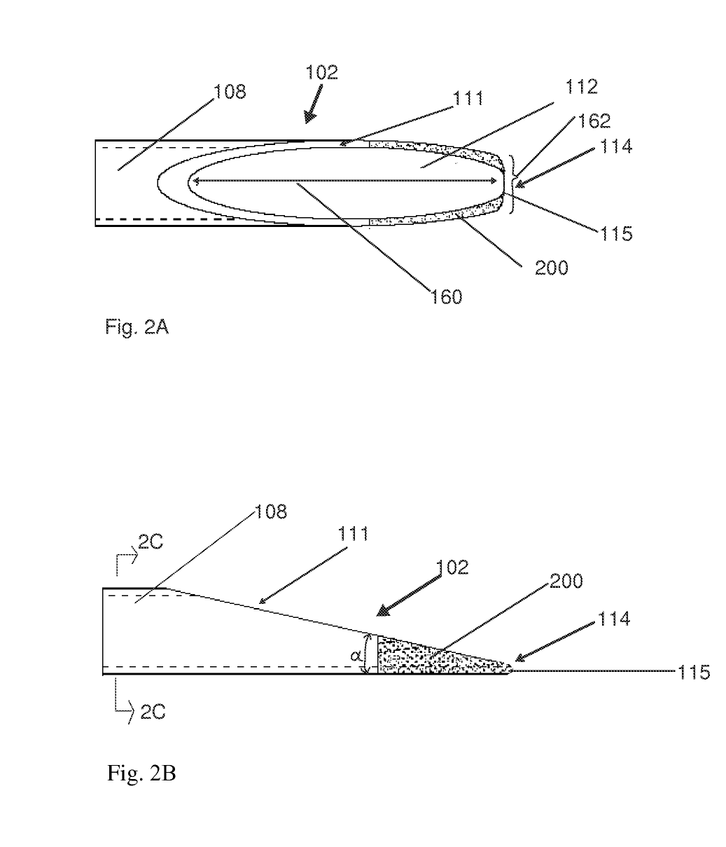

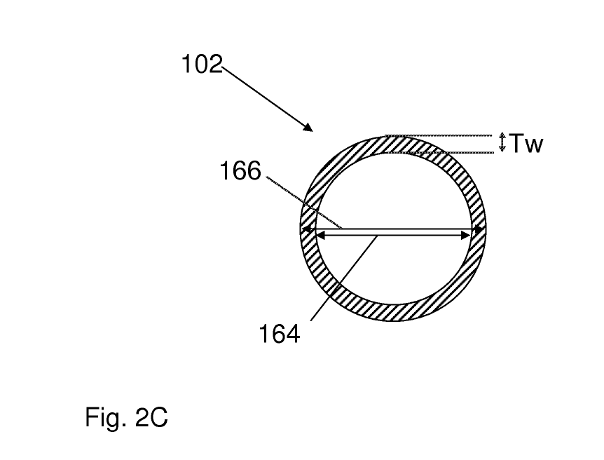

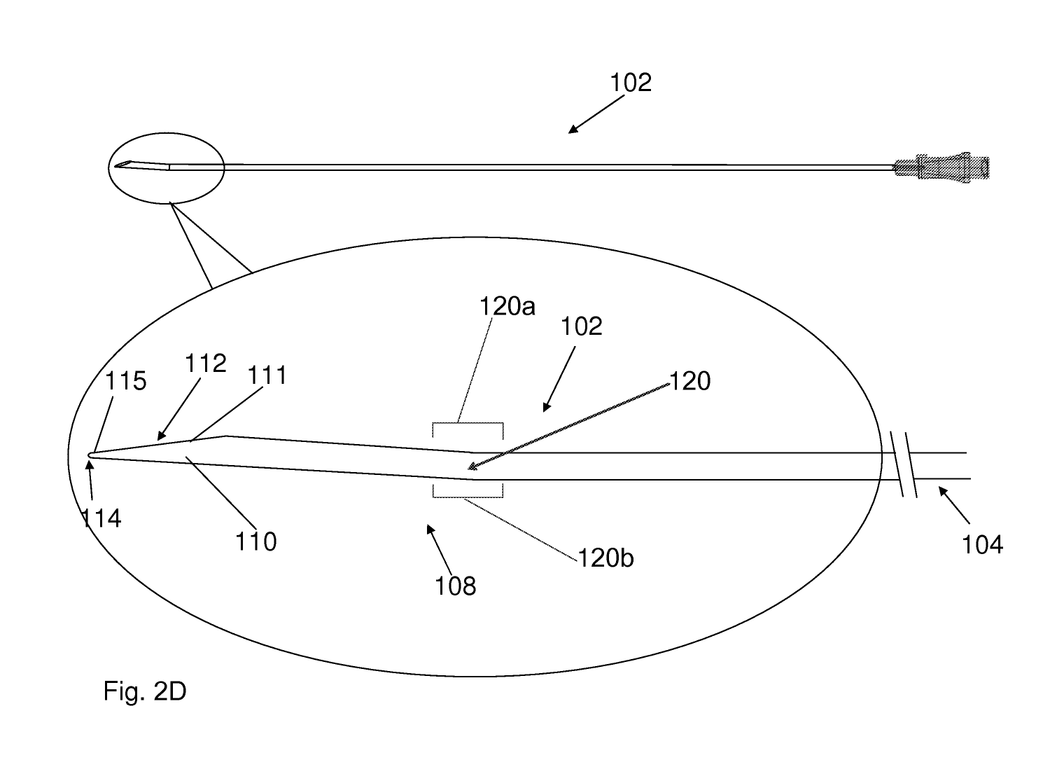

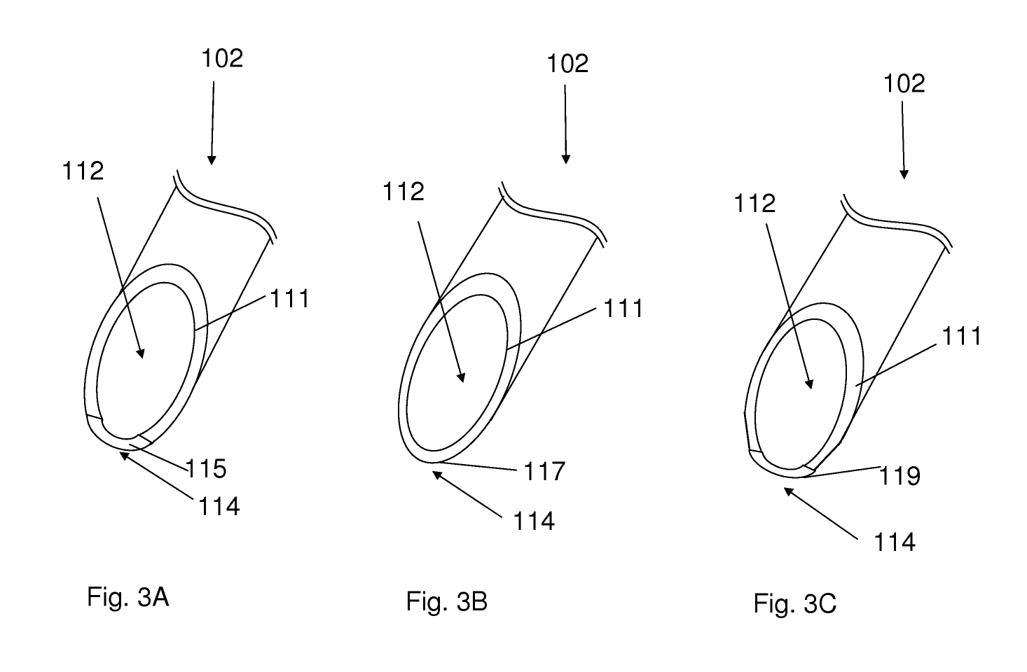

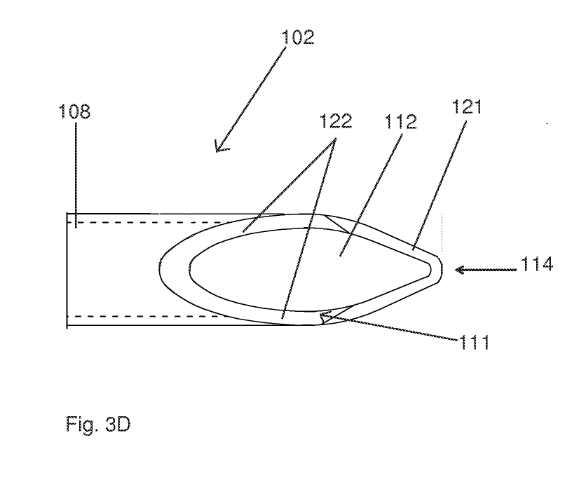

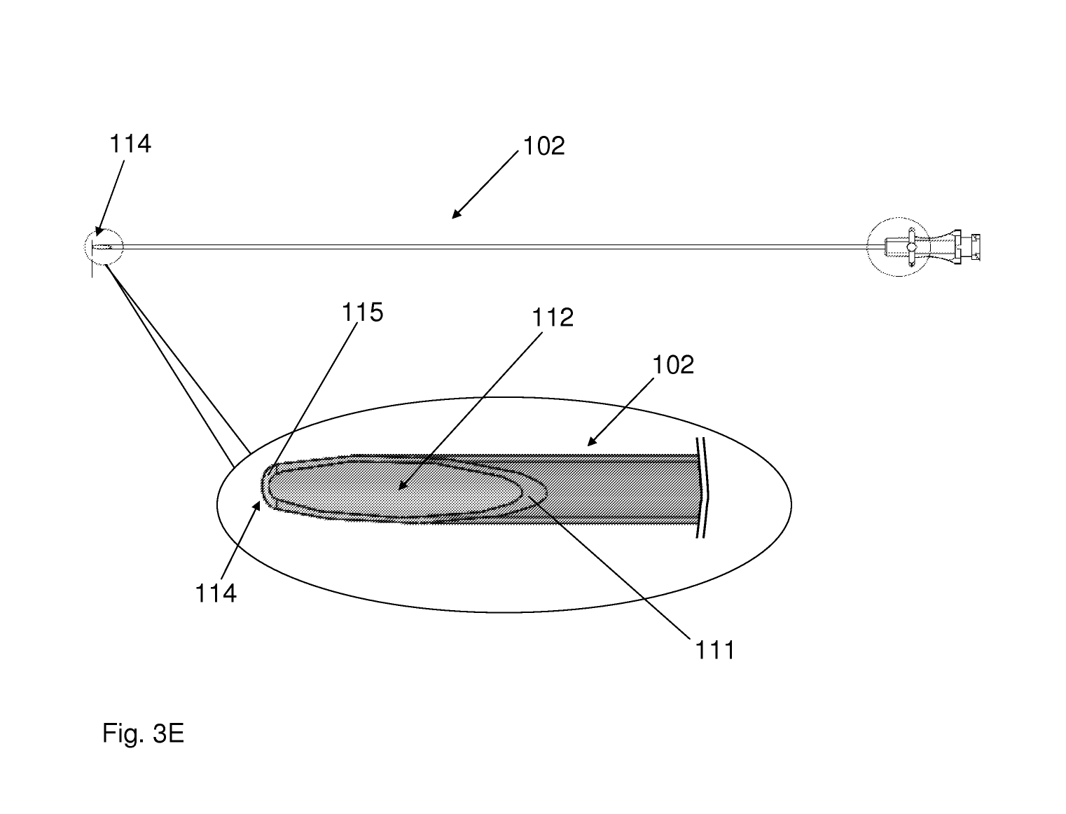

In one embodiment of the present disclosure, the sharp tip 113 of the bevel face 111 has been altered such that it is no longer sharp and the elongate member has a blunt tip 114, as illustrated in FIG. 2A. As discussed herein “blunt tip” refers to a tip that does not have a sharp edge or a point. The blunt tip 114 is dull which means that it does not have the ability to cut easily. The blunt tip 114 reduces the risk of damage to tissue within the patient's body and is substantially atraumatic. Furthermore, the blunt tip 114 has a greater surface area than the sharp tip 113, which increases the force required to push the tip across a region of tissue such as the annulus fibrosus. In one specific embodiment the sharp bevel tip 113 is blunted to a square tip 115, as shown in FIGS. 2A-2B and 3A, 3E. The blunt square tip 115 has a square edge which comprises a flat edge with substantially rounded corners, where the flat edge is substantially perpendicular to longitudinal axis of the elongate member 102. FIG. 2B illustrates a side profile of the square blunt tip 115.

In one example the blunt tip 114 is formed by grit-blasting the tip to dull any sharp edges or points. The sharp tip 113 may otherwise be blunted using any other means. In one example electrochemical polishing may be used to remove material from the sharp bevel tip 113. Electrochemical polishing can polish and smooth out the bevel tip by removing any burrs from the cutting process. In another example, the sharp bevel tip 113 may be rounded by shaving or by sanding. In still another example, the sharp bevel tip 113 may be blunted by using a laser to cut it into shape. In one embodiment as shown in FIGS. 2A and 2B, the distal end of the elongate member 102 has been dulled to a smooth finish. In one specific example the smooth outer surface 200 at the distal end of the elongate member 102 is created through grit-blasting.

In one embodiment, as shown in FIG. 2D, the elongate member 102 may be bent or curved. This may allow for easier access to a target site. The bend or curve may be applied by the user prior to or during the procedure, or may be applied during manufacture. For example, if the target tissue is the nucleus pulposus of an intervertebral disc, the curve may be at such an angle that the posterior portion of the nucleus pulposus may be reached with the device while allowing for an approach that reduces risk of damage to the spinal canal. In one embodiment, the tissue removal apparatus 100 may be used within the intervertebral disc. And the angle of the bend, or in other words the angle of curvature of the elongate member 102 may be between about 0° to about 8.6° with respect to the longitudinal axis of the elongate member 102. More specifically, the angle of curvature may be between about 6.6° and about 8.6°. In one example, the bend has an angle of about 7.6°. In other embodiments, any suitable angle of curvature or bend may be used. Furthermore, the curve or bend may be located at various points along the length of the elongate member 102. In some embodiments, the bevel face 111 may face upwards with the bend 120 as shown in FIG. 2D. In other words the bevel face 111 may be positioned on the same side as the upper side 120a of the bend 120. Whereas, in other embodiments the bevel face 111 may be positioned at the opposite side or the lower side 120b of the bend 120. In still other embodiments the bevel face may be positioned in other orientations with respect to the bend 120. Elongate member 102 may be manufactured from a number of different materials. These include, but are not limited to, stainless steels, shape-memory materials such as nickel titanium alloys, polyesters, polyethylenes, polyurethanes, polyimides, nylons, copolymers thereof, and medical grade plastics. In one specific embodiment, elongate member 102 is made from a clear, transparent or translucent plastic or other material. This embodiment may allow the user to visualize the contents of elongate member 102 to ensure that the elongate member (or any other device disposed within the elongate member) is operating properly. This may allow for visibility in order to see if material (for e.g. tissue) is being conveyed or if there is a blockage. In one specific example, the elongate member 102 may be made from stainless steel. In one embodiment the elongate member 102 may not be bent. In one specific example of this embodiment, the elongate member is made from Nitinol. Nitinol has elastic properties, which may prevent the elongate member 102 from being permanently deformed or bent when force is applied.

As illustrated in FIG. 2A, the bevel face 111 defines an opening 112. The length of the opening 112 along the longitudinal axis of the elongate member 102 is defined as the bevel opening length 160. In some embodiments, the bevel opening length 160 may be generally between about 0.1 inches to about 0.3 inches. In other embodiments any other suitable bevel opening length 160 may be provided. In one specific example, the elongate member 102 comprises a 19 Gauge hypotube and the bevel opening length is about 0.158 inches. In an alternate example, the elongate member 102 comprises an 18 Gauge hypotube and bevel opening length is about 0.185 inches. The blunt tip 114 has a tip length 162. Tip length 162 is defined as the length of the tip along a transverse axis of the elongate member. The tip length 162 ranges from about from about 0.01 to about 0.06 inches. The maximum tip length 162 of the blunt tip 114 is defined by an outer diameter of the elongate member. In one specific example, the tip length 162 is about 0.03 inches. In another example, the tip length 162 is about 0.06 inches. The bevel angle α is the angle defined by the bevel face 111 and the longitudinal axis of a distal portion of the elongate member 102. The bevel angle α shown in FIG. 2B may range from about 10 degrees to about 15 degrees. In one specific example, the bevel angle α is about 12.5 degrees. In alternate embodiments the bevel angle α may be less than about 10 degrees. In other embodiments the bevel angle α may be greater than about 15 degrees.

In one specific embodiment, elongate member 102 is sized to be percutaneously directed to an interior tissue of the body. The length of elongate member 102 is generally between about 5 inches to about 12 inches, however it may be otherwise sized to reach any target tissue within the body. In one specific example, the length of the elongate member is about 5.2 inches. In another example the length is about 8.2 inches. In a still further example the length is about 11.2 inches. As is illustrated in FIG. 2C, the elongate member 102 comprises both an inner diameter 164 and an outer diameter 166. The elongate member 102 may comprise a hypo-tube of between about 14 Gauge to about 20 Gauge. In some embodiments the elongate member 102 may comprise a hypotube of less than about 14 Gauge. In other words, the outer diameter 166 of the elongate member may be between about 0.030 inches to about 0.090 inches; however it may be otherwise sized to fit within the space defined by the target tissue. In some embodiments the outer diameter 166 may be greater than 0.090 inches. In one specific example, the elongate member 102 may comprise a 19 Gauge thin wall hypotube. The inner diameter 164 may be about 0.033 inches and the outer diameter 166 may be about 0.042 inches and the wall thickness T_{w} is about 0.004 inches. In another example, the elongate member 102 may comprise an 18 Gauge thin wall hypotube. The inner diameter 164 may be about 0.042 inches and the outer diameter 166 may be about 0.050 inches and the wall thickness T_{w} of the elongate member 102 may be about 0.004 inches. In other embodiments, the wall thickness T_{w} may be between about 0.0035 inches to about 0.01 inches.

As illustrated in FIGS. 3A-3C, the blunt tip 114 of the bevel 111 may have other configurations. As mentioned above the blunt bevel tip 114 may have a square configuration 115 which is shown in FIGS. 2A-2B and 3A. Alternatively, the blunt tip 114 may have a curved configuration. FIG. 3B illustrates a curved blunt tip 117 that comprises an edge with a curved profile. In an alternate embodiment the blunt tip 114 may have a tapered configuration. FIG. 3C shows a blunt tapered tip 119 which may be formed from a blunt square tip 115 that has been further modified to have tapered side edges. In an alternate embodiment as shown in FIG. 3D, the bevel face 111 may have sharpened lateral edges 122. The blunt tip 114 is a blunt lancet tip 121 that comprises a lancet tip that has then been rounded off at its distal end. The sharpened lateral edges 122 may define a secondary bevel angle. In one specific embodiment, the elongate member 102 may be bent or curved. The elongate member may have a blunt lancet tip 121 and may have sharpened lateral edges 122. The sharpened lateral edges 122 may help facilitate easier movement of the bent elongate member 102 within the nucleus. In other embodiments, the blunt tip 114 may have any other suitable configuration other than the configurations shown herein, that is substantially atraumatic.

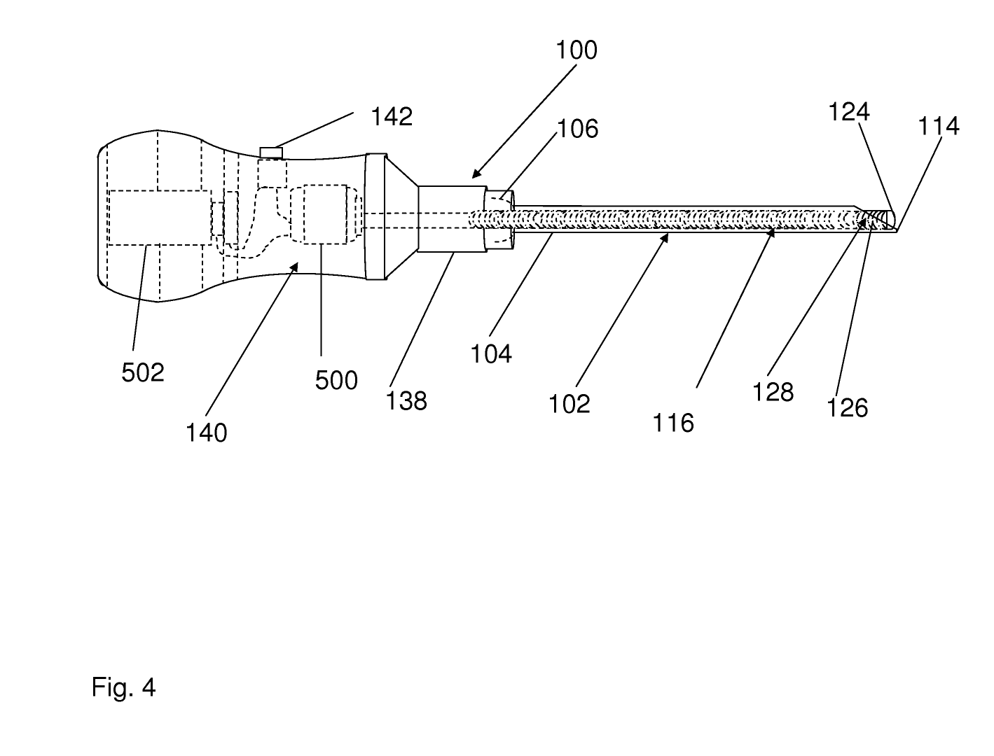

In one embodiment of the present disclosure, for example as shown in FIG. 4, an apparatus 100 is disclosed which comprises an elongate member 102, a tissue removal member 116, a hand piece 140, and a receptacle 138 for collection and/or visualization of tissue. The tissue removal member 116 is structured to be disposed within elongate member 102. Tissue removal member 116 comprises a proximal portion and distal portion ending in distal end 124. In one embodiment, tissue removal member 116 comprises a shaft 128 having outwardly extending projections 126. In one example, the outwardly extending projections may comprise a helical flighting. Alternatively the projections may comprise a helical element disposed at least partially around shaft 128. In one embodiment the outwardly extending projections 126 may comprise a coil that has been helically wound around the shaft 128. In one embodiment, tissue removal member 116 may be coaxial with elongate member 102; however tissue removal member 116 may be otherwise aligned. In a specific embodiment, distal end 124 of tissue removal member 116 is recessed proximally from the blunt distal tip 114 of elongate member 102. This structure allows for the distal portion of tissue removal member 116 to be substantially shrouded within the elongate member 102. In other words the distal end 124 of the tissue removal member 116 is recessed from the blunt distal tip 114 of the elongate member 102 such that the tissue removal member 116 is exposed on one side to the bevel opening, whereas, the tissue removal member 116 is covered or shielded on other sides by the elongate member 102. This arrangement may protect surrounding tissues from being damaged by contact with tissue removal member 116.

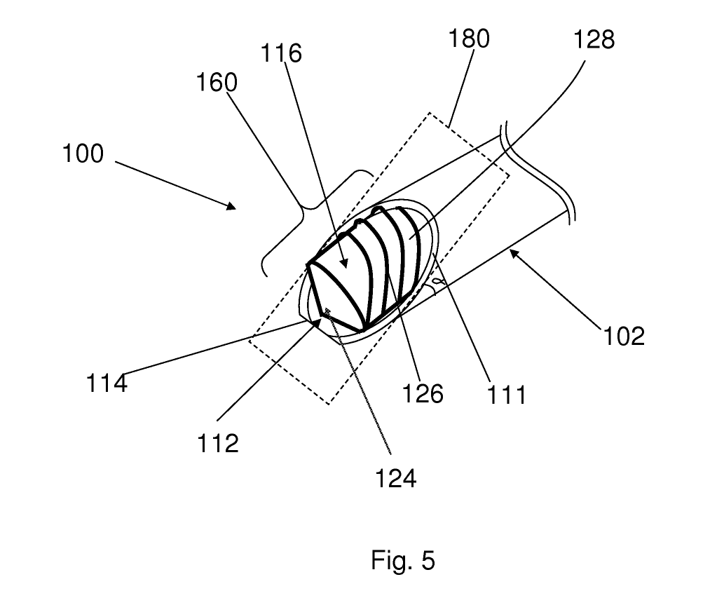

In accordance with one embodiment of the present disclosure, a tissue removal apparatus 100 is disclosed which comprises an elongate member 102 comprising a bevel face 111 as illustrated in FIG. 5. The bevel face 111 has an opening 112 with a bevel opening length 160 and is oriented at a bevel angle α with respect to the longitudinal axis of a distal portion of the elongate member 102. The bevel opening 112 allows the distal portion of the tissue removal member 116 to be exposed to tissue at the distal end of the apparatus 100. As discussed above, the distal end 124 of the tissue removal member 116 is recessed from the blunt distal tip 114 of the elongate member. The distal end 124 is located proximal to the blunt distal tip 114 such that at least a portion of the tissue removal member 116 protrudes beyond the opening 112 defined by the bevel face 111. In other words, the tissue removal member 116 is positioned such that the distal end 124 breaks the plane 180 defined by the opening 112. The tissue removal member 116 is positioned within the elongate member 102 such that at least some of the outwardly extending projections 126 of the tissue removal member 116 are protruding from the opening 112. In one embodiment about 2 to about 5 outwardly extending projections 126 may be exposed. In one embodiment, the outwardly extending projections 126 are formed from a coil that has been helically wound around the shaft 128. In some embodiments about 3 to 4 turns of the helical wire 176 forming the outwardly extending projection 126 may be exposed to the opening 112. In one example, about 3 turns of the helical wire 176 are exposed. In some embodiments less than about 2 turns or greater than about 5 turns may be exposed. The bevel opening length 160 may be maximized by decreasing the bevel angle α which may increase exposure of the tissue removal member 116 to tissue which maximizes the bevel opening length 160. For example, this allows the tissue removal member 116 to access material distal to the opening 112. In other words, the open distal end of the bevel face 111 allows the tissue removal member 116 to be exposed to tissue located distal to the plane defined by the opening 112, as well as tissue located distal to the blunt distal tip 114. In one example, the bevel angle α may be between about 10 degrees to about 15 degrees.

Tissue removal member 116 may generally be between about 6 inches and about 18 inches in length, more specifically between about 8.0 inches and about 15 inches. In one example the length of the tissue removal member 116 is about 9 inches. In one example, the length of the tissue removal member 116 is about 12 inches. In another example the length of the tissue removal member 116 is about 15 inches. The diameter of shaft 128 may generally be between about 0.012 inches and about 0.042 inches, more specifically between about 0.013 inches and about 0.028 inches. The width or thickness of the outwardly extending projections 126 may generally be between about 0.003 inches and about 0.025 inches, more specifically between about 0.005 inches and about 0.010 inches. In one example, the outwardly extending projections 126 are formed from a coil that has been helically wound around the shaft 128. In one example, the shaft 128 has a diameter of about 0.024 inches. A 0.004×0.006 inch coil is helically wound around the shaft 128. The distance or spacing between adjacent outwardly extending projections 126 or the pitch is between about 0.04 inches to about 0.05 inches. In other embodiments, the pitch may range from about 0.001 inches to about 0.1 inches. In one example, the distance between the outer diameter of the shaft and the outer diameter of the coil may be about 0.012 inches.

As previously mentioned the apparatus 100 shown in FIG. 4 may comprise a receptacle or collection chamber, 138 for housing and/or viewing any material, for e.g. tissue that may be removed from the body. The collection chamber 138 is operatively connected to the elongate member 102 for receiving the material that may be removed from the body. The elongate member 102 defines at least one opening for transferring material from the elongate member 102 to the collection chamber 138. In some embodiments, collection chamber may be structured to be coupled to handpiece 140. In yet another embodiment, collection chamber 138 may be located within handpiece 140. In some embodiments, collection chamber 138 may be structured to allow the user to visualize and measure the amount of material such as tissue in the chamber and may be detachable. In one embodiment, the handpiece 140, as shown in FIG. 4, may be structured to house a battery 502, a motor 500, and electrical connections therebetween. The motor 500 may be operatively connected to the shaft of the tissue removal member 116. In addition, a button or switch 142 may be located on handpiece 140.

In some embodiments, tissue removal member 116 may be operatively connected to a source of motorized rotational energy, for example a motor 500, to allow for rotation of tissue removal member 116. In one specific embodiment, motor 500 may be connected to battery 502. When a switch 142 is engaged, motor 500 may cause shaft 128 of tissue removal member 116 to rotate, thereby rotating outwardly extending projections 126 and conveying tissue from the distal portion of tissue removal member 116 to proximal portion. The elongate member 102 may comprise a hub 106 that mates with the distal portion of the handle. In one embodiment the hub 106 of the elongate member 102 comprises luer threads that engage with a luer on the distal portion of the handle. In one specific embodiment, the elongate member 102 is fabricated from Nitinol. The thermal properties of Nitinol may allow for greater heat dissipation, which may help to minimize any changes in temperature resulting from heat generation due to rotation of the tissue removal member 116 within the elongate member 102.

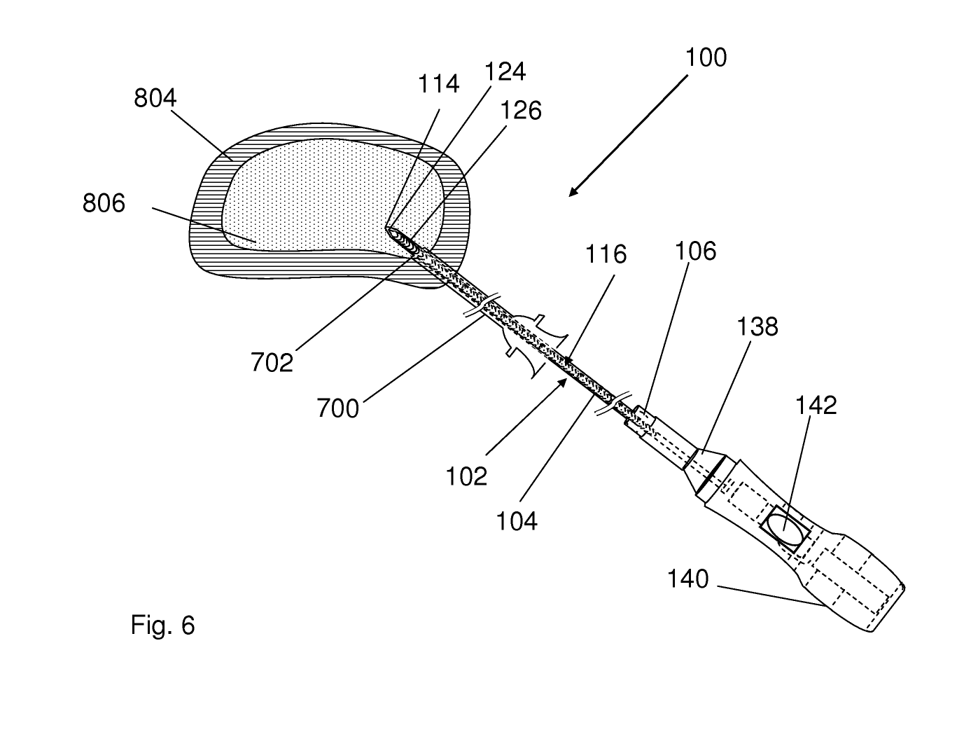

In some embodiments, as shown in FIG. 6, apparatus 100 may comprise an introducer apparatus that will aid in introducing elongate member 102 into the target tissue. The introducer apparatus may include a hollow elongate introducer or cannula 700 and an obturator. Cannula 700 may be substantially stiff or rigid, such that it may assist in piercing skin or other body tissues, or such that it may provide support for apparatus 100. The obturator may be structured to cooperatively engage with cannula 700. In some embodiments the distal tip of the obturator may be sharp and may be conical, beveled, tri-beveled or trochar tipped. Further details regarding the apparatus are provided in U.S. application Ser. Nos. 11/128,342, 11/368,491, 11/368,505, 11/368,506, 11/368,508, 11/368,509, 11/368,510, 11/368,475 and 11/368,513, previously incorporated herein by reference.

In one broad aspect, the disclosure comprises methods for removal of material from a body. The methods described herein may be used to remove various types of materials from a patient's body. Examples of such materials include, but are not limited to, tissue of an intervertebral disc (for example, the nucleus pulposus), tumor tissue (including, but not limited to, material from breast, colon, stomach, or liver tumors), bone tissue (for example, bone marrow), cyst material, adipose tissue, eye material, cartilage, or atherosclerotic material. In one embodiment, the method of the present disclosure may be practiced using apparatus 100, including tissue removal member 116 disposed within elongate member 102, as described hereinabove. In one example, the apparatus 100 may be used as a disc-decompression device. In one embodiment of the present disclosure an introducer apparatus, as illustrated in FIG. 6, comprising a cannula 700 and an obturator (not shown), is advanced until distal end 702 of cannula 700 is positioned at the target site which is the boundary between the annulus fibrosus 804 and the nucleus pulposus 806. The obturator is then withdrawn proximally from cannula 700, leaving a distal end 702 of cannula 700 at the leading annulus wall of the intervertebral disc. The elongate member 102 may then be inserted through the lumen of cannula 700 and advanced until the blunt distal tip 114 of the elongate member 102 is located distal to distal end 702 of cannula 700.

Tissue may then be removed using the electrosurgical device by engaging the motor and activating the tissue removal member in order to remove tissue. The elongate member may be advanced through the target site and material can be conveyed away from the target site. After the desired volume of tissue has been removed the apparatus 100 may be removed from the body, as discussed further below. In one embodiment, tissue removal member 116 is coupled to motor 500. Upon engagement of motor 500, tissue removal member 116 rotates about its longitudinal axis. Outwardly extending projections 126, described hereinabove, will engage the tissue within elongate member 102, and convey the tissue toward proximal end of tissue removal member 116. In one specific embodiment, proximal portion of tissue removal member 116 is operatively connected to collection chamber 138 as described hereinabove. In some embodiments as the apparatus 100 is advanced, it may eventually contact the annulus fibrosis 804. As the tissue removal member 116 is recessed from the blunt distal tip 114 of elongate member 102 the annulus fibrosis 804 may be protected from the high speed motion and projections of tissue removal member 116 by the blunt distal tip 114. The device of the present disclosure may protect the annulus wall by minimizing the risk of puncture or perforation due to the tissue removal member 116 contacting the annulus wall. The tissue removal member is recessed within the elongate member 102 such that the distal end 124 of the tissue removal member 116 lies proximal to the blunt distal tip 114 of the elongate member 102. Thus, the tissue removal member 116 does not come into contact with the annulus fibrosus 804.

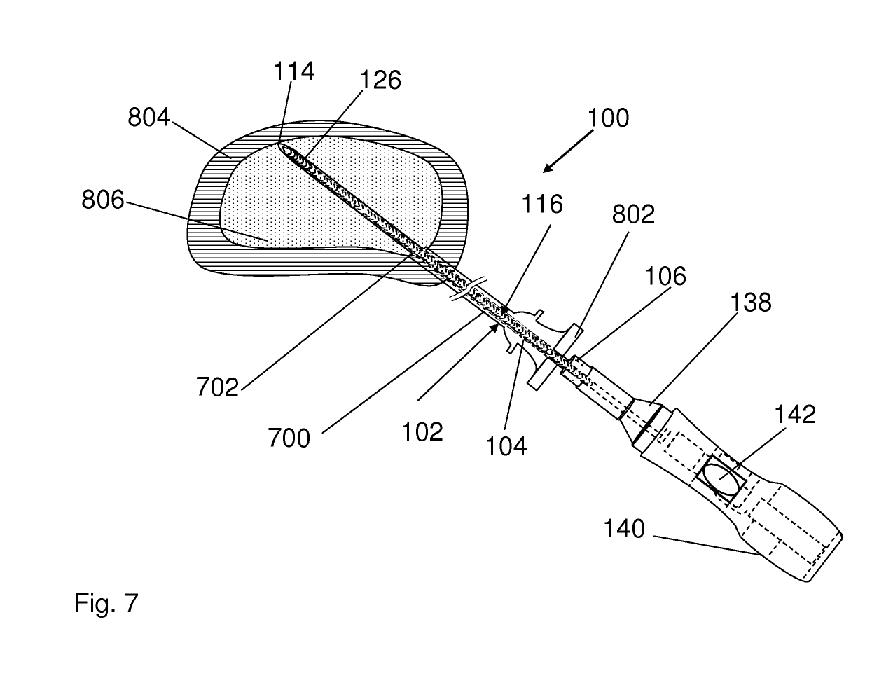

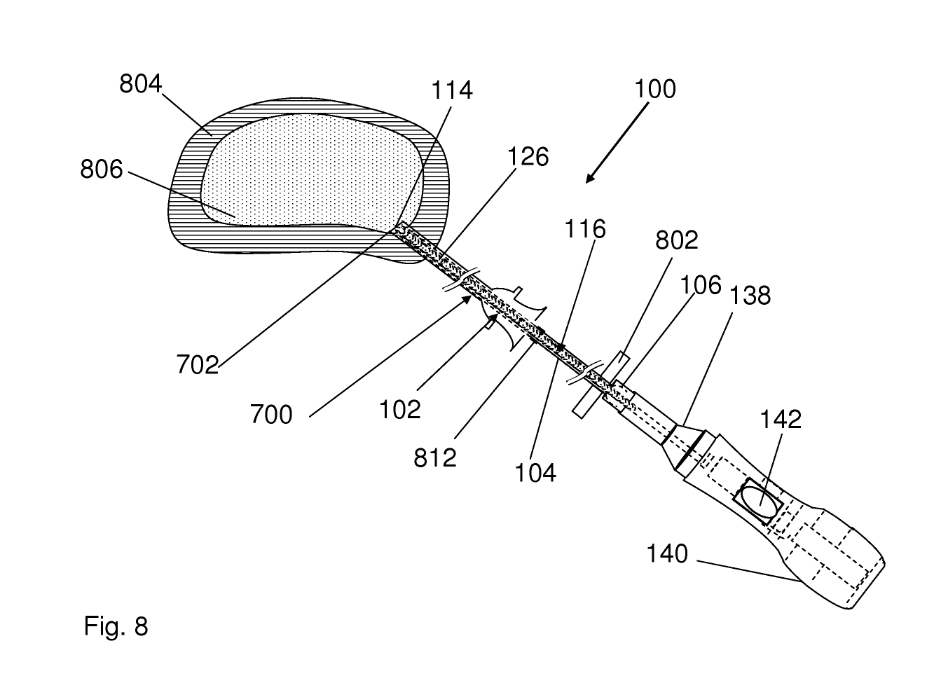

In some embodiments, after the tissue removal apparatus 100 has been positioned at the leading annulus wall, the user may then advance elongate member 102 through nucleus pulposus 806, without activating tissue removal member 116, until the blunt distal tip 114 of elongate member 102 contacts annulus fibrosis 804 on the anterior side or portion of the disc, as shown in FIG. 7. This point of contact may be referred to the as the “anterior annulus inner wall” of the intervertebral disc. The boundary between nucleus pulposus 806 and annulus fibrosis 804 may be located, for example, by tactile sensation, as the annulus fibrosis 804 is generally stiffer than the nucleus pulposus 806, or by using a contrast solution and performing the method under fluoroscopy, as described hereinabove. Once the distal tip end 114 has been positioned at the anterior annulus inner wall, the user may place a marker or depth stopper 802 on the distal most portion of elongate member 102 that is proximal to the proximal end of cannula 700. After the depth stopper 802 has been placed, the user may then retract the blunt distal tip 114 of elongate member 102 proximally to distal end 702 of cannula 700. In one embodiment, proximal portion 104 of elongate member 102 may comprise a marking 812 located such that when the marking is aligned with the proximal end of cannula 700, it indicates that the distal ends of cannula 700 and elongate member 102 are aligned as shown in FIG. 8. Thus, when elongate member 102 is withdrawn proximally through the disc, the user will know to stop retracting elongate member 102 when marking 812 is aligned with the proximal end of cannula 700. The user may then engage tissue removal member 116, and begin the coring and conveyance procedure and may advance the apparatus distally through the disc. As shown in FIG. 7, the user may stop advancing apparatus 100 through the disc as a depth-stopper 802 approaches the proximal end of cannula 700. In an alternate embodiment a marker may be used instead of the depth-stopper 802. The user may then withdraw apparatus and repeat the coring and conveying step. The use of a marker or depth stopper 802 may help to ensure that the distal tip 114 of elongate member 102 does not contact annulus fibrosis 804. This may be especially advantageous in the case of a severely damaged disc that may be severely affected by damage to annulus fibrosis 804.

In one embodiment of the present disclosure, a depth stopper may not be used and the user may advance the apparatus 100 until the blunt distal tip 114 contacts the anterior annulus inner wall as described above. The blunt distal tip 114 limits or prevents the penetration of the blunt distal tip 114 into the annulus fibrosus 804. Thus the blunt bevel tip 114 of the embodiment of the present disclosure ensures that if the distal end of the tissue removal apparatus 100 is inadvertently placed close to the annulus fibrosus 804, any further advancement of the apparatus 100 may not result in an annulus wall tear. Thus, material may be removed from the disc by positioning the blunt distal tip 114 of the elongate member 102 in any location within the disc without significantly compromising the safety of the procedure. Material of the nucleus pulposus 806 may therefore be removed from the disc without compromising the structural integrity of the disc. This may be a significant risk factor in degenerated discs having reduced support within the annulus wall. A problem associated with devices comprising a ‘traumatic’ tip is that the penetration of a tip into the annulus fibrosus 804 can damage the annulus fibers and may create a mechanical tear within the wall. The ‘traumatic’ tip may be created by the use of a sharp tip, or by using a device that has laser or RF at the tip. In some cases, the use of a traumatic tip, for e.g. a sharp tip may initiate a collapse of the disc. Thus, the blunt bevel tip 114 of the present embodiment may prevent unnecessary penetration of the elongate member 102 into tissue of the annulus wall. More specifically it may minimize the risk of complications resulting from puncturing of the annulus fibrosus.

Furthermore, the blunt bevel tip 114 may allow for increased steerability of the elongate member 102. The bevel opening provides an asymmetrical interface with tissue when it is advanced within a region of tissue. This in turn allows forces to be distributed unevenly along the open distal end 110 of the elongate member. As the elongate member is advanced within the tissue, the force of the tissue against the bevel face 111 steers the elongate member in a direction perpendicular to the bevel face 111. This can be used advantageously to avoid any sensitive structures, such as a bone or a nerve, in the path of the tissue removal apparatus 100 as it is advanced. If the distal end of the elongate member is adjusted such that the bevel face 111 faces a sensitive structure, for example a nerve. Then, as the elongate member 102 is advanced within tissue, the blunt distal tip 114 of the elongate member is steered away from the nerve, due to the forces of the tissue acting on the bevel face 111. Once the distal end 110 has been advanced past the sensitive structure, the elongate member 102 can be turned such that the bevel face 111 is facing away from the nerve. This allows the distal end 110 of the elongate member to be steered around the nerve. Thus, the asymmetry of the bevel face 111 increases the steerability of the elongate member 102. This can be advantageous as it can allow a physician to make adjustments to the position or orientation of the distal end 110 of the elongate member 102 without withdrawing the elongate member 102 completely from the patient's body and re-inserting at a desired angle. Thus, changing the orientation of bevel face 111 at any point during advancement of the apparatus may allow the physician to steer the elongate member to adjust the angle at which the apparatus is being advanced.

Thus, the bevel face 111 enhances the exposure of the distal portion of the tissue removal member 116 to tissue at the distal end of the apparatus. This exposes the tissue removal member 116 to tissue that is located both distal to the opening 112 and distal to the blunt distal tip 114. This allows effective removal of tissue from a wider area.

Additionally, the blunt bevel tip 114 provides a safety feature by preventing the tip from penetrating the annulus fibrosus. Furthermore, the embodiments of the present disclosure provide a tissue removal member 116 having a distal end 124 that is recessed from the distal tip 114 of the elongate member 102, which may further minimize the risk of damage to the annulus fibrosus 804 by contact with the tissue removal member 116.

Still furthermore, the bevel face 111 provides asymmetry which allows for selective removal of tissue. The bevel face 111 allows one side of the tissue removal member 116 to be exposed to tissue. Tissue can be collected only from one side of the elongate member 102. In other words, it allows for greater tissue collection from an area from which a tissue sample is desired, while reducing exposure to tissue that does not need to be removed. If the bevel tip 114 is near the annulus, the user may rotate the apparatus 100 to position the blunt tip 114 such that the bevel face 111 is oriented away from the annulus wall 804. This permits the tissue removal member 116 to interface with only the nucleus pulposus 806, decreasing the risk of damage to the annulus fibrosus 804. Thus, the bevel face 111 of the elongate member 102 allows for asymmetry at the disc device interface during use. This can be advantageous in applications where tissue needs to be selectively removed such as for treating a focal herniation. Thus, the asymmetry allows for the apparatus to be positioned for optimum tissue collection in a safer and more controlled fashion. Furthermore, the blunt bevel tip 114 may allow for increased steerability of the elongate member 116. This can allow a physician to steer the elongate member such the distal tip 114 does not come into contact with any sensitive structures. Furthermore, it can allow the distal end 110 elongate member to be steered around a sensitive structure and can also enable adjustments to the angle at which the elongate member 102 is advanced within the patient's body.

In one broad aspect, embodiments of the present disclosure comprise an apparatus for removing tissue from a patient's body. The apparatus comprises a first elongate member defining a lumen and having an open distal end, said open distal end defining an opening and comprising a tip, said tip being structured to reduce damage to tissue within said patient's body. The apparatus further comprises a tissue removal member disposed within said lumen, said tissue removal member having a distal end, said distal end being recessed from said tip, at least a portion of the tissue removal member protruding beyond said opening to allow access to tissue distal to said opening.

As a feature of this aspect, said open distal end comprises a bevel face, said bevel face being at an angle with respect to a longitudinal axis of said elongate member. In one embodiment, the angle may range from about 10 degrees to about 15 degrees. In one example, the angle is about 12.5 degrees.

As another feature of this aspect, said tip is a blunt tip. In one example the blunt tip is a square blunt tip. In another example, the blunt tip is a curved blunt tip. In another example, the blunt tip is a tapered blunt tip. In still another example, the blunt tip is a blunt lancet tip.

The embodiments of the disclosure described above are intended to be exemplary only. The scope of the disclosure is therefore intended to be limited solely by the scope of the appended claims.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure.

We claim:

1. An apparatus for removing tissue from a patient's body comprising:
a first elongate member defining a lumen and having an open distal end, said open distal end defining an opening and comprising a tip, said tip structured to reduce damage to tissue within said patient's body; and
a tissue removal member disposed within said lumen, said tissue removal member having a distal end, said distal end being recessed from said tip, at least a portion of the tissue removal member protruding beyond said opening to allow access to tissue distal to said opening.

2. The apparatus of claim 1, wherein said open distal end comprises a bevel face, said bevel face being at an angle with respect to a longitudinal axis of said elongate member.

3. The apparatus of claim 2, wherein said tip is a blunt tip.

4. The apparatus of claim 3, wherein said blunt tip comprises a square blunt tip.

5. The apparatus of claim 3, wherein said blunt tip comprises a curved blunt tip.

6. The apparatus of claim 3, wherein said blunt tip comprises a tapered blunt tip.

7. The apparatus of claim 3, wherein said blunt tip comprises a blunt lancet tip.

8. The apparatus of claim 2, wherein said angle ranges from about 10 degrees to about 15 degrees.

9. The apparatus of claim 8, wherein said angle is about 12.5 degrees.

10. The apparatus of claim 1, wherein said tissue removal member comprises a plurality of outwardly extending projections for conveying tissue through said lumen, at least some of said plurality of outwardly extending projections protruding from said opening of the elongate member distal end.

11. The apparatus of claim 10, wherein said outwardly extending projections form at least one helix.

12. The apparatus of claim 12 further comprising a motorized source of rotational energy operatively connected to said tissue removal member.

13. The apparatus of claim 11, further comprising a handpiece operatively connected to said tissue removal member.

14. The apparatus of claim 13, further comprising a motorized source of rotational energy operatively connected to said tissue removal member, wherein said handpiece comprises means for engaging and disengaging said motorized source of rotational energy.

15. The apparatus of claim 11, further comprising a collection chamber operatively connected to the elongate member for receiving the tissue removed from the body, said elongate member defining at least one opening for transferring material from said elongate member to said collection chamber.

16. The apparatus of claim 15, wherein said receptacle is detachable from said elongate member.

17. A hollow member for insertion into a patient's body to receive a tissue removal member for removing tissue via the hollow member, the hollow member comprising:
an elongate member defining a lumen and having an open distal end having a bevel face, said bevel face being at an angle with respect to a longitudinal axis of said elongate member, said open distal end defining an opening and having a blunt tip, said blunt tip being structured for advancement within the patient's body to the site of tissue to be removed, the presence of the blunt tip structure reducing damage to tissue within said patient's body during advancement.

18. The hollow member of claim 17, wherein said angle ranges from about 10 degrees to about 15 degrees.

19. The hollow member of claim 18, wherein said angle is about 12.5 degrees.

20. The hollow member of claim 18, wherein said blunt tip comprises a blunt lancet tip.

21. The hollow member of claim 18, wherein said blunt tip comprises a square blunt tip.

22. The hollow member of claim 18, wherein said blunt tip comprises a tapered blunt tip.

23. The hollow member of claim 18, wherein said blunt tip comprises a curved blunt tip.

24. The hollow member of claim 18, wherein said blunt tip is configured with a grit-blasted surface.
